(19) United States
(12) Patent Application Publication
(10) Pub. No.: US 2023/0364064 A1
(43) Pub. Date: Nov. 16, 2023

(54) USE OF TETRANDRINE IN COMBINATION WITH ALL-TRANS RETINOIC ACID IN PREPARATION OF MEDICAMENT FOR TREATING PNEUMOCONIOSIS

(71) Applicant: Institute of Basic Medical Sciences of Chinese Academy of Medical Sciences, Beijing (CN)

(72) Inventors: Jing WANG, Beijing (CN); Meiyue SONG, Beijing (CN); Jiaxin WANG, Beijing (CN)

(73) Assignee: Institute of Basic Medical Sciences of Chinese Academy of Medical Sciences, Beijing (CN)

(21) Appl. No.: 18/063,695

(22) Filed: Dec. 9, 2022

(30) Foreign Application Priority Data
May 13, 2022 (CN) 202210524196.9

(51) Int. Cl. A61K 31/4375; A61K 31/203; A61P 11/00

(52) U.S. Cl. CPC A61K 31/4375; A61K 31/203; A61P 11/00

(57) ABSTRACT

The present disclosure is directed to methods of administering tetrandrine in combination with all-trans retinoic acid (ATRA) for treating pneumoconiosis. Administration of ATRA in combination with tetrandrine can effectively alleviate the progression of silicosis: impaired cardiopulmonary functions of mice with silicosis are improved significantly, including inspiratory capacity, quasi-static compliance, and right ventricular pressure; concentrations of inflammatory factors IL-1β and IL-6 in bronchoalveolar lavage fluids of mice with silicosis decrease, and the number of inflammatory cells is reduced; levels of fibrosis factors FN-1 and Col-I decrease, fibrous foci are reduced, and pathological degree is alleviated. Moreover, combination therapy with ATRA in combination with tetrandrine shows a superiority of combined administration in terms of cardiopulmonary function, inflammation and fibrosis, and exhibits a better effect than administration of tetrandrine alone.

### CROSS REFERENCE TO RELATED APPLICATION(S)

This application claims the benefit and priority of Chinese Patent Application No. 202210524196.9, filed on May 13, 2022, the disclosure of which is incorporated by reference herein in its entirety as part of the present application.

### TECHNICAL FIELD

The present disclosure relates to the field of biomedicine, in particular to use of tetrandrine in combination with all-trans retinoic acid (ATRA) in the preparation of a medicament for treating pneumoconiosis.

### BACKGROUND ART

Pneumoconiosis is an occupational pulmonary disease manifesting as diffuse pulmonary fibrosis, caused by long-term inhalation of pathogenic produced dust in occupational activities and retention in the lungs, and is one of the most important occupational diseases in the world. Basic lesions of pneumoconiosis include acute and chronic pulmonary inflammation, irreversible pulmonary fibrosis, and pulmonary dysfunction or failure at the advanced stage of the disease, and specific pathogenesis thereof remains unclear. At present, it is believed that pulmonary fibrosis of pneumoconiosis is unrecoverable. The existing therapeutic approaches only delay the progression of fibrosis. Therefore, pneumoconiosis is still a disease without medical endpoint.

Anti-fibrosis is the core of the treatment of pneumoconiosis. But unfortunately, there are no effective anti-fibrotic methods and drugs for treating pneumoconiosis inside and outside of China. At present, interventions that are commonly used clinically include bronchoalveolar lavage and lung transplantation. Bronchoalveolar lavage can scavenge a large amount of pathogenic dust deposited in the lungs, inflammatory cells, and pro-inflammatory and pro-fibrogenic factors, alleviate patients' symptoms, and improve impairment of lung function, but there is a lack of evidence-based evidence of reversal of fibrosis and long-term outcome. Patients with late pneumoconiosis are mostly accompanied with progressive/massive fibrosis, which leads to respiratory failure and low quality of life; a minority of patients can undergo lung transplantation to prolong life. However, lung transplantation has problems of scarcity of donor, high surgery cost, and long-term poor prognosis, and cannot be widely used clinically. Medications for pneumoconiosis mainly include cough-suppressing and phlegm-transforming agents, anti-inflammatory and anti-asthmatic agents, and other symptomatic agents, as well as anti-silicosis fibrosis drugs, including tetrandrine (Tet) and Xifeining Pian (tablets). Of them, Tet has been used in China for more than 50 years and has become a recommended drug for diagnosis and treatment consensus by pneumoconiosis experts in China in 2018. However, the efficacy of Tet in the treatment of pneumoconiosis is not desired. Therefore, it is necessary to look for a new therapeutic regimen.

### SUMMARY

In view of this, an objective of the present disclosure is to provide use of Tet in combination with ATRA in the preparation of a medicament for treating pneumoconiosis. Administration of Tet in combination with ATRA is effective against the pneumoconiosis and satisfies a desired outcome.

To achieve the above objective, the present disclosure provides the following technical solutions:

The present disclosure provides use of Tet in combination with ATRA in the preparation of a medicament for treating pneumoconiosis.

Preferably, the pneumoconiosis may include silicosis, coal worker's pneumoconiosis, graphite pneumoconiosis, carbon black pneumoconiosis, asbestosis, talc pneumoconiosis, cement pneumoconiosis, mica pneumoconiosis, kaolin pneumoconiosis, aluminosis, arc-welders pneumoconiosis, and foundry workers' pneumoconiosis.

Preferably, the medicament may include individually packaged Tet and ATRA.

Preferably, when the medicament is used to treat mice with pneumoconiosis, a Tet solution may be administered intragastrically at a dose of 60 mg/kg, 6 times per week, for 4 weeks;
an ATRA solution may be administered intragastrically at a dose of 10 mg/kg, 3 times per week, for 4 weeks.

Preferably, the Tet solution may use CMC-Na as a solvent and have a concentration of 10 mg/mL
the ATRA solution may use corn oil as a solvent and have a concentration of 2.5 mg/mL.

The present disclosure further provides a medicament for treating pneumoconiosis, including individually packaged Tet and ATRA.

Preferably, the medicament may be in the pharmaceutical dosage form of a powder, a tablet, or a solution.

Beneficial effects: The present disclosure provides use of Tet in combination with ATRA in the preparation of a medicament for treating pneumoconiosis. In the example, a mouse model of silicosis is used for studying. Administration of ATRA in combination with Tet to mice with silicosis can effectively alleviate the progression of silicosis, specifically: after administration of Tet in combination with ATRA, impaired cardiopulmonary functions of mice with silicosis are improved significantly, including inspiratory capacity, quasi-static compliance, and right ventricular pressure; concentrations of inflammatory factors IL-1β and IL-6 in bronchoalveolar lavage fluids of mice with silicosis decrease, and inflammatory cell aggregation is reduced; levels of fibrosis factors FN-1 and Col-I decrease, fibrous foci are reduced, and pathological degree is alleviated. Moreover, combination therapy of ATRA in combination with Tet shows a superiority of combined administration in terms of cardiopulmonary function, inflammation and fibrosis, and exhibits a better effect than administration of Tet alone. Therefore, administration of ATRA in combination with Tet can be used as a new therapeutic regimen for pneumoconiosis.

### BRIEF DESCRIPTION OF THE DRAWINGS

FIG. 1A-1J illustrates the effect of administration of ATRA in combination with Tet on silicosis, where panel A is a schematic diagram showing an experiment for the treatment of silicosis by administration of ATRA in combination with Tet; panels B and C illustrate mouse lung function, where IC represents inspiratory capacity and Cst represents quasi-static compliance; panel D illustrates mouse right ventricular function, where RVSP represents right ventricular systolic pressure; panel E illustrates concentrations of IL-1β in mouse bronchoalveolar lavage fluid, where BALF represents the bronchoalveolar lavage fluid; panel F illustrates HE staining (top) and Masson staining (bottom) of mouse lung tissues; panel G illustrates concentrations of IL-6 in mouse BALF; panels H and I illustrates translational and transcriptional levels of Fn-1 in mouse lung tissues; panel J illustrates the transcriptional level of Col-I in mouse lung tissues; **P<0.01, ***P<0.001.

### DETAILED DESCRIPTION OF THE EMBODIMENTS

In the following description, numerous specific details are set forth, such as particular structures, components, materials, dimensions, processing steps and techniques, in order to provide an understanding of the various embodiments of the present application. However, it will be appreciated by one of ordinary skill in the art that various embodiments of the present application may be practiced without these specific details. In other instances, well-known structures or processing steps have not been described in detail in order to avoid obscuring the present application.

As used herein, the term “substantially” or “substantial”, is equally applicable when used in a negative connotation to refer to the complete or near complete lack of an action, characteristic, property, state, structure, item, or result. For example, a surface that is “substantially” flat would either be completely at, or so nearly flat that the effect would be the same as if it were completely flat.

As used herein, terms defined in the singular are intended to include those terms defined in the plural and vice versa.

As used in this specification and its appended claims, terms such as “a”, “an” and “the” are not intended to refer to only a singular entity, but include the general class of which a specific example may be used for illustration, unless the context dictates otherwise. The terminology herein is used to describe specific embodiments of the disclosure, but their usage does not delimit the disclosure, except as outlined in the claims.

Unless otherwise indicated, all numbers expressing quantities of ingredients, properties such as molecular weights, reaction conditions, and so forth as used in the specification and claims are to be understood as being modified in all instances by the term “about.” Accordingly, unless indicated to the contrary, the numerical parameters in the specification and claims are approximations that may vary depending upon the desired properties sought to be obtained by the present disclosure. At the very least, and without limiting the application of the doctrine of equivalents to the scope of the claims, each numerical parameter should at least be construed in light of the number of reported significant digits and by applying ordinary rounding techniques. Notwithstanding that the numerical ranges and parameters describing the broad scope of the disclosure are approximations, the numerical values in the specific examples are reported as precisely as possible. Any numerical value, however, inherently contains standard deviations that necessarily result from the errors found in the numerical value's testing measurements.

Thus, reference herein to any numerical range expressly includes each numerical value (including fractional numbers and whole numbers) encompassed by that range. To illustrate, reference herein to a range of “at least 50” or “at least about 50” includes whole numbers of 50, 51, 52, 53, 54, 55, 56, 57, 58, 59, 60, etc., and fractional numbers 50.1, 50.2 50.3, 50.4, 50.5, 50.6, 50.7, 50.8, 50.9, etc. In a further illustration, reference herein to a range of “less than 50” or “less than about 50” includes whole numbers 49, 48, 47, 46, 45, 44, 43, 42, 41, 40, etc., and fractional numbers 49.9, 49.8, 49.7, 49.6, 49.5, 49.4, 49.3, 49.2, 49.1, 49.0, etc. In yet another illustration, reference herein to a range of from “5 to 10” includes whole numbers of 5, 6, 7, 8, 9, and 10, and fractional numbers 5.1, 5.2, 5.3, 5,4, 5,5, 5.6, 5.7, 5.8, 5.9, etc.

In the discussion and claims herein, the tern “about” indicates that the value listed may be somewhat altered, as long as the alteration does not result in nonconformance of the process or structure to the illustrated embodiment. For example, for some elements the term “about” can refer to a variation of ±0.1%, for other elements, the term “about” can refer to a variation of ±1% or ±10%, or any point therein.

Reference now will be made in detail to embodiments of the disclosure. It will be apparent to those skilled in the art that various modifications and variations can be made in the present disclosure without departing from the scope or spirit of the disclosure. For instance, features illustrated or described as part of one embodiment can be used on another embodiment to yield a still further embodiment.

The present disclosure provides use of Tet in combination with ATRA in the preparation of a medicament for treating pneumoconiosis.

Types of the pneumoconiosis are not particularly limited in the present disclosure, and all types of the pneumoconiosis may be diagnosed based on the “diagnostic criteria for pneumoconiosis” and “pathological diagnostic criteria for pneumoconiosis”, and fall within the protection scope of the present disclosure, preferably including silicosis, coal worker's pneumoconiosis, graphite pneumoconiosis, carbon black pneumoconiosis, asbestosis, talc pneumoconiosis, cement pneumoconiosis, mica pneumoconiosis, kaolin pneumoconiosis, aluminosis, arc-welders pneumoconiosis, and foundry workers' pneumoconiosis.

In the present disclosure, the medicament may preferably include individually packaged Tet and ATRA. In the example of the present disclosure, when pneumoconiosis mouse models are administered intragastrically, a Tet solution may be administered intragastrically at a dose of 60 mg/kg once a day, 6 days per week, for 4 weeks; an ATRA solution may be administered intragastrically (2.5 mg/mL) at a dose of 10 mg/kg, 3 times per week, on alternate days, 6 days per week, for 4 weeks. In the present disclosure, the Tet solution may preferably use CMC-Na as a solvent and have a concentration of 10 mg/mL; the ATRA solution may preferably use corn oil as a solvent and have a concentration of 2.5 mg/mL. In the present disclosure, when the intragastric administration is conducted, both drugs may be administered in combination, namely, in the same week, the Tet solution may preferably be administered intragastrically for six consecutive days and suspended for one day per week, while the ATRA solution may be administered intragastrically for three consecutive days and every other day per week.

In the example, a mouse model of silicosis is used for studying. Administration of ATRA in combination with Tet to mice with silicosis can effectively alleviate the progression of silicosis, specifically: after administration of Tet in combination with ATRA, impaired cardiopulmonary functions of mice with silicosis are improved significantly, including inspiratory capacity, quasi-static compliance, and right ventricular pressure; concentrations of inflammatory factors IL-1β and IL-6 in bronchoalveolar lavage fluids of mice with silicosis decrease, and inflammatory cell aggregation is reduced; levels of fibrosis factors FN-1 and Col-I decrease, fibrous foci are reduced, and pathological degree is alleviated. Moreover, combination therapy of ATRA in combination with Tet shows a superiority of combined administration in terms of cardiopulmonary function, inflammation and fibrosis, and exhibits a better effect than administration of Tet alone.

The present disclosure further provides a medicament for treating pneumoconiosis, including individually packaged Tet and ATRA. Pharmaceutical dosage forms of the medicament are not particularly limited in the present disclosure, preferably including a powder, a tablet, or a solution.

The use of Tet in combination with ATRA in the preparation of a medicament for treating pneumoconiosis provided by the present disclosure will be described in detail below with reference to the examples, but they should not be construed as limiting the protection scope of the present disclosure.

### Example 1

Administration of ATRA in combination with Tet treats silicosis effectively.

1.1 Construction of a Mouse Model of Silicosis and Administration of ATRA in Combination with Tet

Male C57BL/6J mice (aged 8-10 weeks and weighing 25-30 g) were selected and housed in an SPF grade laboratory animal room, and the model of silicosis was constructed by one-time intratracheal instillation of silica (Si); the mice were divided into four groups (n=9):
(1) PBS+Vehicle group: 40 μL of sterile phosphate-buffered saline (PBS) was administered by intratracheal instillation; two weeks later, corn oil was administered intragastrically 3 times a week, 6 days a week, for 4 weeks; 1% CMC-Na was administered intragastrically once a day, 6 days a week, for 4 weeks;
(2) Si+Vehicle group: a silica suspension (300 mg/mL, 40 μL) was administered by intratracheal instillation; two weeks later, corn oil was administered intragastrically 3 times a week, 6 days a week, for 4 weeks; 1% CMC-Na was administered intragastrically once a day, 6 days a week, for 4 weeks;
(3) Si+Tet group: a silica suspension (300 mg/mL, 40 μL) was administered by intratracheal instillation; two weeks later, corn oil was administered intragastrically 3 times a week, and Tet (prepared with 60 mg/kg 1% CMC-Na, 10 mg/mL) was administered intragastrically once a day, 6 days a week, for 4 weeks;
(4) Si+ATRA+Tet group: a silica suspension (300 mg/mL, 40 μL) was administered by intratracheal instillation; two weeks later, ATRA (prepared with 10 mg/kg corn oil, 2.5 mg/mL) was administered intragastrically every other day, 3 times a week, and Tet (60 mg/kg, 10 mg/mL) was administered intragastrically once a day, 6 days a week, for 4 weeks. All mice were sacrificed after 6 weeks, and the corresponding samples were collected for detection.

### 1.2 Cardiopulmonary Function Test

An anesthetized mouse was fixed on an experimental bench, and an 8-gauge needle connected with a high-fidelity pressure sensor was inserted into the right ventricle to directly measure the invasive right ventricular systolic pressure (RVSP). The RVSP was recorded and analyzed by a Power Lab data acquisition and analysis system (PL 3504, AD Instruments, Australia). Mouse lung function was detected by a pulmonary function testing system (DSI Buxco, USA). Before experiment, the mouse was anesthetized by intraperitoneal injection of 0.4 mL/100 g 2% pentobarbital, tracheotomy was performed, a trachea cannula was inserted, and a ventilator was connected. Next, FRC, PV, FV, and RC were automatically tested by a PET system. A statistical analysis was conducted on indexes closed related to lung function changes of silicosis, including inspiratory capacity (IC) and quasi-static compliance (Cst).

### 1.3 Pathological Staining

The left lung was fixed in 4% paraformaldehyde for 24 h, dehydrated, paraffin-embedded, sectioned (to 5 μm), and subjected to HE staining and Masson staining, respectively. For HE staining, inflammation was scored based on Szapiel scores; for Masson staining, the severity of fibrosis was scored by Aschroft scores. The sections were scanned, photographed and counted by a 3D HISTECH digital slide scanner.

### 1.4 ELISA

Concentrations of inflammatory factors IL-1β and IL-6 in mouse BALF were detected by using ELISA kits.

### 1.5 Western Blot

Lung tissue proteins of all mice were extracted. A 10% polyacrylamide gel was prepared, and 10 μg of protein was loaded and electrophorese at 80 V for 30 min; after the voltage was adjusted to 120 V, the electrophoresis was continued until it ended. Polyvinylidene fluoride (PVDF) membrane was used for the transfer and blocked with 5% skimmed milk powder for 1 h, the primary antibody FN-1 (abcam, 1:1,000) was incubated at 4° C. overnight and the secondary antibody was incubated at room temperature for 1 h, and finally blots were developed by chemiluminescence. Then, the band was incubated with β-actin antibody. The remaining steps were identical as described above.

1.6 qPCR

Lung tissue RNA of all mice was extracted; cDNA was obtained by using a reverse transcription kit (KR103, Tiangen Biotechnology, Beijing, China); qPCR was conducted by using a SYBR Green I Q-PCR Kit (TransGen Biotech, Beijing China); data collection and analysis were conducted by Bio-Rad IQ5 system.

TABLE 1
Primer sequences (5′ to 3′)

| | | | |
|---|---|---|---|
| β-actin | F | TAGGCACCAGGGTGTGAT | SEQ ID NO: 1 |
| | R | CTCCTCAGGGGCCACA | SEQ ID NO: 2 |
| Fn-1 | F | GACGAAGAGCCCTTACAGTTCCA | SEQ ID NO: 3 |
| | R | TCTGCAGTGCCTCCACTATG | SEQ ID NO: 4 |
| Col-I | F | CCTGGTCCCTCTGGAAATG | SEQ ID NO: 5 |
| | R | GGAAGCCTCTTTCTCCTCTC | SEQ ID NO: 6 |

### 1.7 Result Analysis

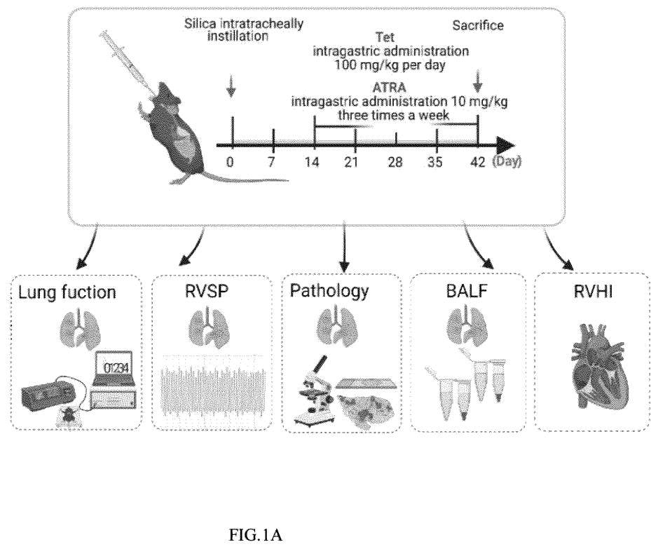

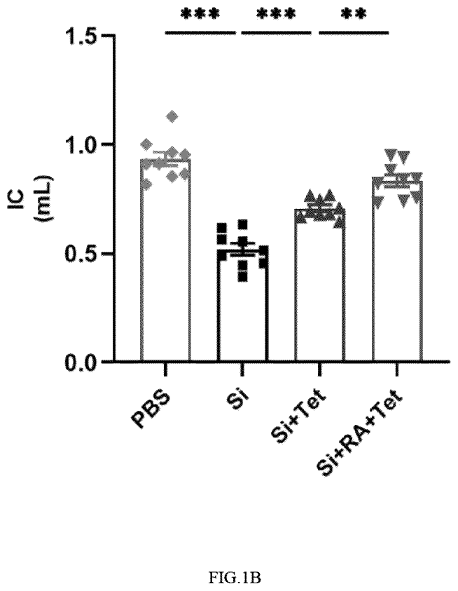

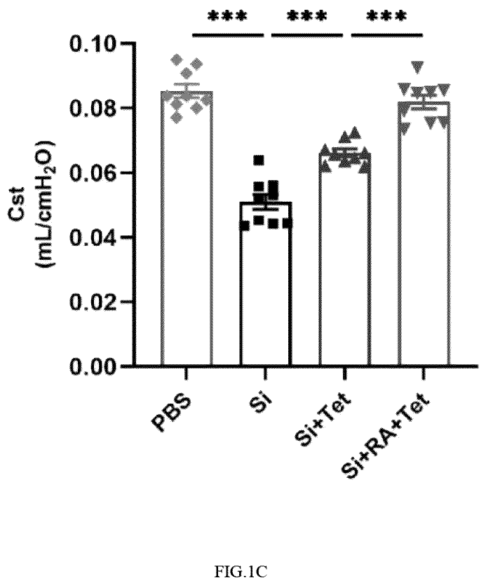

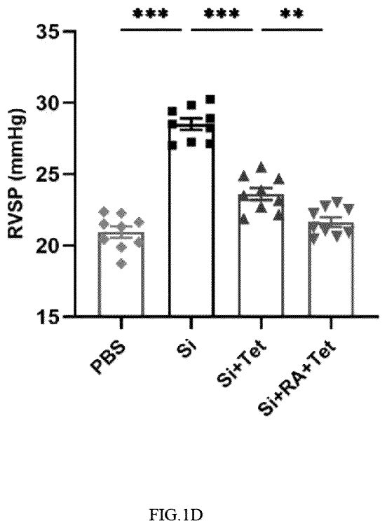

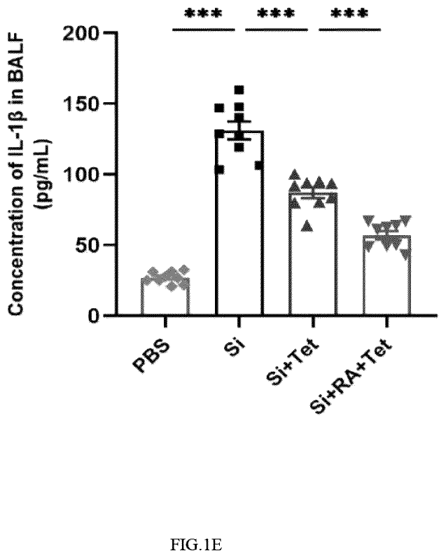

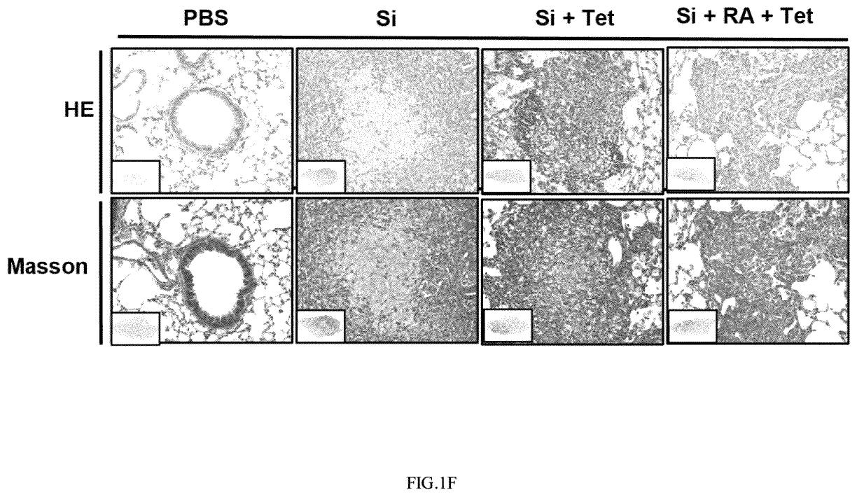

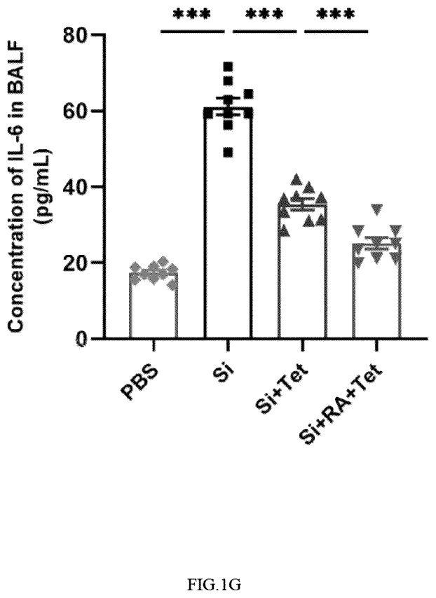

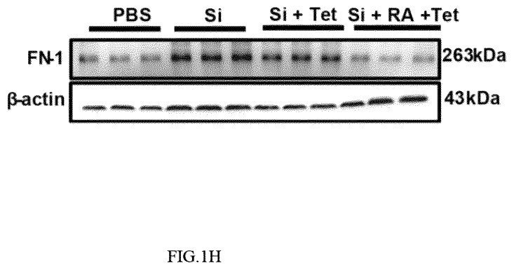

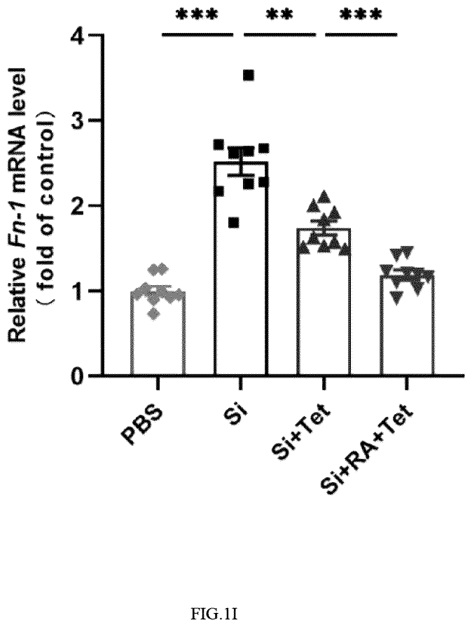

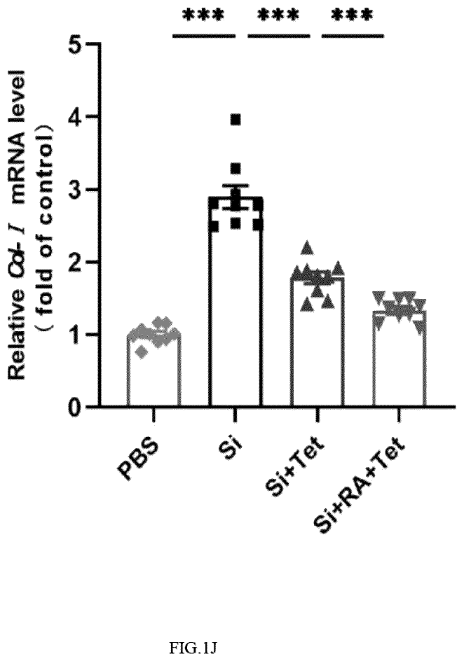

The results are shown in FIG. 1A-J. Administration of ATRA in combination with Tet to mice with silicosis can effectively alleviate the progression of silicosis. After administration of ATRA in combination with Tet, impaired cardiopulmonary functions of mice with silicosis are improved significantly, including IC (FIG. 1B), Cst (FIG. 1C), and RVSP (FIG. 1D). In the aspect of inflammation, administration of both drugs in combination reduces concentrations of inflammatory factors IL-1β (FIG. 1E) and IL-6 (FIG. 1F) in BALF of mice with silicosis, and HE staining (FIG. 1F) also shows a decrease in inflammatory cell aggregation. In the aspect of fibrosis, administration of ATRA in combination with Tet can reduce levels of fibrosis factors FN-1 (FIGS. 1H and 1I) and Col-I (FIG. 1J), and Masson staining (FIG. 1F) shows that fibrous foci are reduced. Moreover, combination therapy of ATRA in combination with Tet shows a superiority of combined administration in terms of cardiopulmonary function, inflammation and fibrosis, and exhibits a better effect than administration of Tet alone.

The above descriptions are merely preferred implementations of the present disclosure. It should be noted that a person of ordinary skill in the art may further make several improvements and modifications without departing from the principle of the present disclosure, but such improvements and modifications should be deemed as falling within the protection scope of the present disclosure.

```
    Sequence Listing Information:
      DTD Version: V1_3
      File Name: GWP20220400045.xml
      Software Name: WIPO Sequence
      Software Version: 2.0.0
      Production Date: 2022-08-03
    General Information:
      Current application / Applicant file reference: GWP20220400045
      Earliest priority application / IP Office: CN
      Earliest priority application / Application number: 202210524196.9
      Earliest priority application / Filing date: 2022-05-13
      Applicant name: Institute of Basic Medical Sciences of the Chinese
Academy of Medical Sciences
      Applicant name / Language: en
      Invention title: USE OF TETRANDRINE IN COMBINATION WITH
ALL-TRANS RETINOIC ACID IN PREPARATION OF MEDICAMENT FOR
TREATING PNEUMOCONIOSIS ( en )
      Sequence Total Quantity: 6
    Sequences:
      Sequence Number (ID): 1
      Length: 18
      Molecule Type: DNA
      Features Location/Qualifiers:
            - source, 1..18
                  > mol_type, other DNA
                  > note, Primer beta-actin-F
                  > organism, synthetic construct
      Residues:
      taggcaccag ggtgtgat 18
      Sequence Number (ID): 2
      Length: 16
      Molecule Type: DNA
      Features Location/Qualifiers:
            - source, 1..16
                  > mol_type, other DNA
                  > note, Primer beta-actin-R
                  > organism, synthetic construct
      Residues:
      ctcctcaggg gccaca 16
      Sequence Number (ID): 3
      Length: 23
      Molecule Type: DNA
      Features Location/Qualifiers:
            - source, 1..23
                  > mol_type, other DNA
                  > note, Primer Fn-1-F
                  > organism, synthetic construct
      Residues:
      gacgaagage ccttacagtt cca 23
      Sequence Number (ID): 4
      Length: 20
      Molecule Type: DNA
      Features Location/Qualifiers:
            - source, 1..20
                  > mol_type, other DNA
                  > note, Primer Fn-1-R
                  > organism, synthetic construct
      Residues:
      tctgcagtgc ctccactatg 20
      Sequence Number (ID): 5
      Length: 19
      Molecule Type: DNA
      Features Location/Qualifiers:
            - source, 1..19
                  > mol_type, other DNA
                  > note, Primer Col-I-F
                  > organism, synthetic construct
      Residues:
      cctggtccct ctggaaatg 19
      Sequence Number (ID): 6
      Length: 20
      Molecule Type: DNA
      Features Location/Qualifiers:
            - source, 1..20
                  > mol_type, other DNA
                  > note, Primer Col-I-R
                  > organism, synthetic construct
      Residues:
      ggaagcctct ttetcctctc 20
END
```

1. A method for treating pneumoconiosis in a mammal, the method comprising:
administering a medicament, the medicament comprising tetrandrine and all-trans retinoic acid (ATRA).

2. The method of claim 1, wherein the pneumoconiosis comprises silicosis, coal worker's pneumoconiosis, graphite pneumoconiosis, carbon black pneumoconiosis, asbestosis, talc pneumoconiosis, cement pneumoconiosis, mica pneumoconiosis, kaolin pneumoconiosis, aluminosis, arc-welders pneumoconiosis, and foundry workers' pneumoconiosis.

3. The method of claim 1, wherein the medicament comprises individual packages of tetrandrine and individual packages of ATRA.

4. The method of claim 1, wherein when the mammal is a mouse with pneumoconiosis, wherein the administration step comprises administration of tetrandrine as a tetrandrine solution, intragastrically, at a dose of about 60 mg/kg, about 6 times per week, for about 4 weeks, and the administration step comprises administration of ATRA as an ATRA solution, intragastrically, at a dose of about 10 mg/kg, about 3 times per week, for about 4 weeks.

5. The method claim 4, wherein the tetrandrine solution comprises CMC-Na as a solvent, wherein the tetrandrine in the CMC-Na solution has a concentration of about 10 mg/mL;
the ATRA solution comprises corn oil as a solvent, wherein the ATRA in the corn oil solution has a concentration of about 2.5 mg/mL.

6. A composition configured to treat pneumoconiosis, the composition comprises:
individually packaged tetrandrine; and
individually packaged ATRA.

7. The composition according to claim 6, wherein the individually packaged tetrandrine and the individually packaged ATRA are each in the form of a powder, a tablet, or a solution.


SEQUENCE LISTING (6 sequences)

SEQ ID NO: 1 - DNA, 18 bp, synthetic construct
  source 1..18: mol_type = other DNA; note = Primer beta-actin-F
taggcaccagggtgtgat

SEQ ID NO: 2 - DNA, 16 bp, synthetic construct
  source 1..16: mol_type = other DNA; note = Primer beta-actin-R
ctcctcaggggccaca

SEQ ID NO: 3 - DNA, 23 bp, synthetic construct
  source 1..23: mol_type = other DNA; note = Primer Fn-1-F
gacgaagagcccttacagttcca

SEQ ID NO: 4 - DNA, 20 bp, synthetic construct
  source 1..20: mol_type = other DNA; note = Primer Fn-1-R
tctgcagtgcctccactatg

SEQ ID NO: 5 - DNA, 19 bp, synthetic construct
  source 1..19: mol_type = other DNA; note = Primer Col-I-F
cctggtccctctggaaatg

SEQ ID NO: 6 - DNA, 20 bp, synthetic construct
  source 1..20: mol_type = other DNA; note = Primer Col-I-R
ggaagcctctttctcctctc